(19) United States
(12) Patent Application Publication
(10) Pub. No.: US 2016/0015306 A1
(43) Pub. Date: Jan. 21, 2016

(54) METHOD FOR DETERMINING AN INDIVIDUAL'S PSYCHOLOGICAL TYPE

(71) Applicant: Aleksandr Petrovich Khudiakov, Novosibirsk (RU)

(72) Inventor: Aleksandr Petrovich Khudiakov, Novosibirsk (RU)

(21) Appl. No.: 14/770,841

(22) PCT Filed: Oct. 28, 2014
(86) PCT No.: PCT/RU2014/000814

§ 371 Date: Aug. 27, 2015

(30) Foreign Application Priority Data
Nov. 21, 2013 (RU) 2013151981

(51) Int. Cl. A61B 5/16; A61B 5/00; A61B 5/11; A61B 7/00; A61B 5/107

(52) U.S. Cl. CPC A61B 5/167; A61B 7/00; A61B 5/107; A61B 5/1114; A61B 5/7246

(57) ABSTRACT

The present invention relates to the field of psychology and medicine and can be used for determination the psyche type of an individual with higher reliability of determination of the results.

The method of determination the psyche type of an individual is in the following: forming a sampling of individuals with different known types of psyche, then selecting and measuring the external parameters, then comparing the selected and measured external parameters of the tested individual with the same external parameters of an individual from sampling, and finally relating psyche type of the tested individual to psyche type of an individual from sampling having similarity by external parameters.

The technical result, achieved by the present invention is in providing higher reliability of the received results in determination the psyche type of an individual.

The present invention relates to psychology and medicine and can be used for determination the psyche type of an individual with higher reliability of results definition.

### PRIOR ART

There are similar disclosures, e.g. see the description of invention to the patent of the Russian Federation [symbol] 2203619, which contains the following set of essential features:
define values of poles (characteristics) of psyche type of the tested individuals by four mutually orthogonal scales:
intuition-logic;
process-state;
facts-relations;
extroversion-introversion,
   these mutually orthogonal scales characterize individual's preferable type of information accordingly integral or discrete, dynamic or statistical, descriptive or evaluative, concrete or abstract;
define the type of personality of the tested individual by combination of poles (characteristics).

The common features of the present invention and the characterized similar invention are:
determination the psyche type of an individual.

There is also another similar disclosure, e.g. see the description of invention to the patent of the Russian Federation [symbol] 2201131, which contains the following set of essential features:
select and measure the external parameters of individuals in the form of amplitudes of oscillatory processes, occurring in the right and left hemispheres of the individuals' cerebrum;
define differences of amplitudes of oscillatory processes, occurring in the right and left hemispheres of the individuals' cerebrum;
define the degree of concordance of these oscillatory processes by means of estimation of their coherence;
define the values of external parameters corresponding to the types of psyche:
stable, if any of hemispheres doesn't dominate and the concordance of oscillatory processes in both hemispheres is high;
unstable, if any of hemispheres doesn't dominate and the concordance of oscillatory processes in both hemispheres is low;
logic, if the left hemisphere dominates and the concordance of oscillatory processes in both hemispheres is average;
intuitive, if the right hemisphere dominates and the concordance of oscillatory processes in both hemispheres is average;
logic stable, if the left hemisphere dominates and the concordance of oscillatory processes in both hemispheres is high;
logic unstable, if the left hemisphere dominates and the concordance of oscillatory processes in both hemispheres is low;
intuitive stable, if the right hemisphere dominates and the concordance of oscillatory processes in both hemispheres is high;
intuitive unstable, if the right hemisphere dominates and the concordance of oscillatory processes in both hemispheres is low;
measure the external parameters of the tested individual;
define psyche type of the tested individual by means of relating the values of individual's external parameters and the external parameters defined for the one or another type of psyche.

The common features of the present invention and the above characterized invention are:
selection of external parameters of individuals;
measure of external parameters of the tested individual;
determination the psyche type of an individual.

There is yet another similar disclosure, e.g. see the description of invention to the patent of the Russian Federation [symbol] 2355293, which contains the following set of features:
individuals are being tested by means of multiple questioning with the help of cards;
the received answers of individuals are estimated;
define the psyche type of individuals by received results and in accordance with types of information metabolism (TIM).

The common features of the present invention and the invention characterized in the patent of the Russian Federation [symbol] 2355293 are:
determination the psyche type of individuals.

There is yet further similar disclosure, published on the website http://psychotype.ru and entitled as “the Classifier of external parameters”, which contains the following set of essential features:
select the external parameters of individuals;
set the generalized values of external parameters corresponding to some or other types of psyche of individuals;
measure the external parameters of the tested individual;
compare the values of external parameters of the tested individual with the generalized values, set for one or another type of psyche;
define the psyche type of the tested individual;

The common features of the present invention and the above characterized disclosure are:
selection of external parameters of individuals;
measure of external parameters of the tested individuals;
determination the psyche type of an individual.

The technical result, which is impossible to achieve by any of characterized similar disclosures, consists in providing higher reliability of received results while defining the psyche type of an individual and it's based on the significant similarity of external parameters of some individuals with close psychic structure of personality.

The reason why the mentioned technical result cannot be achieved is that the existent methods of defining the psyche type with regard to external parameters of individuals relate the values of external parameters of tested individuals to some generalized values, set for different types of psyche.

Unlike the disclosure of prior art, the claimed herein method does not set the generalized values of external parameters of individuals for certain types of psyche, but instead external parameters of tested individuals are compared directly with external parameters of individuals from the comparative sampling of individuals who has different known types of psyche.

The method is based on the observation that individuals with high level of similarity of external parameters have more likely the same psyche type, while the opposite is not true.

Individuals with identical values of separate characteristics and even their pairs in the whole do not have similarity expressed enough, i.e. the attempts to reveal the common external parameters of all sensing will not be effective. Moreover even individuals with coincident values of all characteristics of psyche type do not have external similarities expressed enough. But among individuals with coincident type of psyche there are groups of individuals with similarities of external parameters highly expressed. Therefore the direct comparison of the tested individual with individuals from sampling who have different known types of psyche will be much more effective.

Thus, in the existing disclosure of prior art (e.g. see “the Classifier of external parameters” on the website http://psychotype.ru) an individual with rather large face by characteristic “intuition-sensation” will be more likely defined as “sensing” according to the basic parameter “intuition-sensation”. So, for the intuitive individuals (those who have “intuition” according to characteristic “intuition-sensation”) with rather large faces as well as for the sensing individuals with rather narrow faces the accuracy of determination their psyche type is considerably reduced.

At the same time the claimed herein method allows to define the psyche type of the tested individual with high level of accuracy, in case if there are individuals in comparative sampling with values of external parameters close to those of the tested individual, as far as the similarity level of values of external parameters of the tested individual and individuals in comparative sampling will be estimated directly, rather than by means of the set generalized values of external parameters.

The existing disclosures of prior art allow to project just common tendencies in values of external parameters of individuals with one or another type of psyche and at best can explain the common pattern of this interrelation, than create full-fledged instrument for determination the psyche type.

The claimed herein method of determination the psyche type does not assume any limitations of number of “type portraits” or standard sets of features or range of values of external parameters for every psyche type, as far as the values of external parameters are not being compared with templates (set generalized values) of external parameters, but instead with potentially unlimited number of variants of values of external parameters for every psyche type, existed in reality and included into a comparative sampling.

Nevertheless the revealing of such templates is possible on the basis of the claimed herein method, but in fact their usage for determination the psyche type means comparison of values of external parameters of the tested individual with values of external parameters of one or several individuals from comparative sampling on the basis of weight of external parameters, characterizing their similarity between themselves (i.e. we attach bigger weight for external parameters, whose values are typical for a group of individuals, while smaller weight goes for external parameters with less typical values).

In fact the revealing of generalized external parameters of the psyche types is open scientific problem for current moment, and all existing disclosures of prior art regarding it (including above-mentioned disclosure) are not well-grounded enough for determination the psyche types. The claimed herein method allows to pass over this unsolved scientific problem by means of forming a sampling of real individuals (instead of set generalized parameters), which allow to solve the problem of the psyche type determination, without waiting for the mentioned scientific problem have been solved or probably this method will even favor its solving.

Considering the characteristic and analysis of any known similar disclosures, it is possible to conclude that the problem by creating the methods of determination the psyche types of an individual with higher reliability of results is topical for today.

### SUMMARY OF THE INVENTION

In the claimed herein method of determination the psyche type of an individual the above-mentioned technical result is being achieved by means of forming a sampling of individuals with different known types of psyche, then selecting and measuring the external parameters of the tested individual and individuals from sampling, then comparing the selected and measured external parameters of the tested individual with the same external parameters of individuals from sampling, and then relating psyche type of the tested individual with psyche type of an individual from sampling having similarity by external parameters.

The comparison of the selected and measured external parameters of the tested individual with the same external parameters of individuals from sampling and relating psyche type of the tested individual to psyche type of an individual from sampling with bigger similarity by external parameters allows determining the psyche type of the individual with higher reliability.

The comparison of the selected and measured external parameters of the tested individual with the same external parameters of individuals from sampling and relating psyche type of the tested individual to psyche type of an individual from sampling with bigger similarity by external parameters allows fast determining the psyche type of the individual. Since in the process of comparing external parameters of the tested individual with the same external parameters of individuals from sampling, the enough number of coincidences of external parameters is possible during the first comparisons already, then it is possible to define the psyche type of the individual without further comparison.

It is possible to form a sampling of individuals with different known types of psyche defined on the basis of characteristics ethics-logic, intuition-sensation, extraversion-introversion, and rationality-irrationality. On the basis of the conducted studies the above-mentioned characteristics of psyche types are more connected with external parameters of an individual (see table 1 and 2).

It is possible to form a sampling of individuals with different known types of psyche defined on the basis of characteristics ethics-logic, intuition-sensation, extraversion-introversion, rationality-irrationality and type of temperament. On the basis of the conducted studies the above-mentioned characteristics of psyche types are more connected with external parameters of an individual (see table 1 and 2).

The conducted analysis of similar disclosures of the prior art showed that none of them contains the full set of essential features of the present invention as well as distinguishing features. All this makes possible to conclude that the claimed herein invention meets the requirement of patentability such as “novelty” and “inventive steps”.

The technical main point of the claimed herein method of determination the psyche type of an individual is the following:

The method of determination the psyche type of an individual is in
forming sampling of individuals with different known types of psyche
selecting and measuring the external parameters of the tested individual and individuals from sampling
comparing the selected and measured external parameters of the tested individual with the same external parameters of individuals from sampling,
and relating psyche type of the tested individual with psyche type of an individual from sampling having similarity by external parameters.

### PREFERRED EMBODIMENT OF THE INVENTION

The practical method of determination the psyche type of an individual is the following:

1) It forms a sampling of individuals with different known types of psyche

The requirement for a group of individuals to be seen as a sampling is the presence of at least one individual, because in case of revealing quite high similarity of external parameters of the tested individual and an individual from sampling, the type of the tested individual can be seen defined without additional comparison. Such approach may be required, e.g. to search in a large group of individuals at least one individual with the same psyche type as the individual in the initial sampling. More common case means that there is at least by one individual for each psyche types in the group.

The claimed herein method allows using any typology of psyche. In the example (Table [symbol] 1) the psyche type is defined first on the basis of characteristics: Rationality (R)—irrationality (I), Logic (L)—ethics (E), Intuition (I)—sensation (S), Extroversion (E)—Introversion (I) and second on type of temperament (sanguine (s), choleric (c), phlegmatic (p), melancholic (m)).

The condition of sampling sufficiency: for a concrete definite set of external parameters a sampling will be sufficient, if it provides the method accuracy not less than given level by user. Thus, sampling efficiency can be viewed in relation to the set of external parameters and the level of method accuracy. The level of method accuracy can be defined as the percentage of rightly determined psyche types of the tested individuals.

The method accuracy is increasing together with a number of individuals in a sampling and also with a number of individuals of each psyche type. While selecting individuals for comparative sampling it is necessary to take into consideration factors that can impact the external parameters of individuals independently (age, sex, race and nationality, overweight, diseases, significant defects of appearance or voice, plastic surgery, etc.).

Determination the psyche type (in the example it consists of socionic dichotomies and type of temperament) of individuals in sampling is done by one or several of known methods, e.g. by means of tests, observation, expert estimation by means of interview, etc. In the example the psyche type of the individual number one is defined as RLSIs: rational logic-sensing introvert, sanguine person (values of socionic dichotomies are rationality, logic, sensation, introversion, type of temperament is sanguine). The psyche type of the rest individuals in the comparative sampling was determined by the same way.

2) It selects and measures external parameters of individuals. The external parameters mean any parameters which characterize the individual and which can be perceived by other individuals and/or can be revealed by means of technical, medicine or diagnostic procedures.

The examples of such parameters together with the list of objects of comparison and possible values and data for comparison are given by sections in the table [symbol] 2—statistic external parameters which mainly describes visually perceived objects in permanent state, dynamic external parameters which characterize changes of visually perceived objects (mainly mime, body and body parts movement) and speech external parameters which characterize speech sounds and all related to them. The number of external parameters taking into consideration is not limited. The measuring of values of parameters can be done by means of quantitative measuring of objects of comparison as well as by means of typization/classification of objects, also it can be done by score of expert method, etc. In the example in table [symbol] 1 the following external parameters were selected and measured: the ratio of face width to height; facial angle (face inclination angle relative to the vertical) measured in degrees; nose shape; the change frequency of direction of gaze (number of times/min); the mimic patterns, i.e. presence or lack of typical movements/deformations of expression or their combinations; the speech rate (signs/min); the speech pattern, i.e. presence or lack of one or another typical speech characteristics.

3) It compares external parameters of the tested individual with an individual from sampling. The aim of current stage is to estimate the level of similarity by every separately considered external parameters of the tested individual with the same external parameters of individuals from comparative sampling. The estimation can be done by formalized methods, including algorithmic ones, (e.g. method of estimation of chances according to normal or other probable type of probabilistic distribution of values of parameters), as well as by non-formalized methods, e.g. by means of expert estimation of comparisons of values. In the example the results of comparison is presented by estimations of level of parameters similarity, where 2 points value the closest to the tested individual, 1 point values the one after him and 0 point is for the rest.

4) It relates psyche type of the tested individual to an individual from sampling with similarity by external parameters. The aim of this stage is to estimate the level of similarity of individuals in the whole based on estimation of the level of similarity by every separately considered value of external parameters of individuals. One of the methods of realization of such estimation is weighting system which takes into consideration the significance of one or other external parameters.

In the non-formalized variant such estimation can be received, e.g., by means of the expert method based on the estimation of similarity level of parameters' values. Note that the estimation of the similarity level of individuals and the selection of the closest individual(s) according to the similarity level of external parameters can be done by several stages, and the weight of same external parameters on different stages can be different.

The additional or alternative condition of comparison can be the acceptance of certain level of similarity of external parameters as the sufficient one, and when this level is achieved during the comparison of the tested individual with individuals from sampling the further comparison stops, the psyche type is considered to be coincident with the one of an individual from sampling if the similarity level of external parameters has achieved the given level.

In the current example all points received by individuals (paragraph [symbol] 4) are being weighed relative to the weight, defined by expert method. The following was taken as the additional condition: the similarity of not less 80% of external parameters between the tested individual and individual with bigger similarity of external parameters must be estimated by a sample, including not less 5 randomly selected individuals, not less than 1 point, and the similarity of not less 50% of external parameters must be estimated at 2 points.

An individual [symbol] 5 has got the maximum weighed estimate by the similarity level of external parameters close to the tested individual. And 6 out of 7 (85.7%) of external parameters have 2 points, so the additional condition about the sufficient similarity of external parameters of current individuals is considered to be completed.

The individual [symbol] 5 was defined as the closest to the tested individual by external parameters and he has the known psyche type with values of characteristics: Irrationality, Ethics, Intuition, Introversion, Melancholic temperament, so, the psyche type of the tested individual relates to the same psyche type.

The tested individual after determination the psyche type can be included into a comparative sampling, and it will increase the accuracy of further tests.

In the result of comparison of the selected and measured external parameters of the tested individual with the same external parameters of an individual from sampling, and then relating psyche type of the tested individual to psyche type of an individual from sampling with similarity by external parameters it's possible to define the psyche type of the tested individual.

At the same time the claimed herein method of determination the psyche type of an individual by means of comparison of the selected and measured external parameters of the tested individual with the same external parameters of an individual from sampling, and then relating psyche type of the tested individual to the psyche type of an individual from sampling with bigger similarity by external parameters allow achieving higher reliability of the received results in determining the psyche type of an individual.

### INDUSTRIAL APPLICABILITY

The present invention can become widely used in the field of psychology, medicine, and can be used for determination of psyche type of an individual with higher reliability of determination of the results, thus, it corresponds to the requirement of the industrial applicability.

TABLE No 1

| | | Numbers of the selected individuals | | | | | | Tested |
|---|---|---|---|---|---|---|---|---|
| | Parameter | 1 | 2 | 3 | 4 | 5 | 6 | Individuals |
| Characteristics of | Rationality/Irrationality | R | I | I | R | I | I | — |
| Psyche type | Logic/Ethics | L | L | E | E | E | L | — |
| | Intuition/Sensation | S | S | I | I | I | S | — |
| | Extraversion/Introversion | I | E | I | E | I | I | — |
| | Type of temperament | s | s | c | c | m | c | — |
| Psyche type | | RLSIs | ILSEs | IEIIc | REIEc | IEIIm | ILSEc | ? |
| External | Ratio of face width to | 0.9 | 0.87 | 0.7 | 0.75 | 0.8 | 0.8 | 0.79 |
| Parameters | height | | | | | | | |
| | Facial angle, degrees | 77 | 78 | 81 | 87 | 83 | 80 | 84 |
| | Nose shape | large | straight | turned | aquiline | long | straight | Straight |
| | | | | up | | | | |
| | Frequency of changes | 35 | 60 | 45 | 70 | 28 | 75 | 30 |
| | of direction of gaze | | | | | | | |
| | (number of times/min) | | | | | | | |
| | Mimic patterns | Not | Not | Being | Smiling | Not | Being | Not reveal |
| | | reveal | reveal | surprised, it | it | reveal | surprised, it | |
| | | | | raises | screws | | raises | |
| | | | | eyebrows & | up | | an | |
| | | | | stares | eyes | | eyebrow | |
| | | | | wide- | | | | |
| | | | | eyed | | | | |
| | Speech rate (signs/min) | 180 | 450 | 850 | 950 | 220 | 620 | 200 |
| | Speech patterns | Long | Long | Not | Being | Answering | Not | Answering |
| | | pauses | pauses | reveal | nervous | it fills | reveal | it fill |
| | | before | before | | it speaks | pauses | | pauses |
| | | answering | answering | | till | with sound | | with sound |
| | | | | | exhaustion | “erm” | | “hm” |
| | | | | | of air | | | |
| | | Estimation of similarity level of parameters | | | | | | Value of |
| | Parameter | 1 | 2 | 3 | 4 | 5 | 6 | parameter |
| External | Ratio of face width to | 0 | 0 | 0 | 1 | 2 | 2 | 10 |
| Parameters | height | | | | | | | |
| | Facial angle | 0 | 0 | 1 | 1 | 2 | 0 | 7 |
| | Nose shape | 0 | 2 | 0 | 0 | 1 | 2 | 6 |
| | Frequency of changes | 2 | 0 | 0 | 0 | 1 | 0 | 3 |
| | of direction of gaze | | | | | | | |
| | Mimic patterns | 0 | 0 | 0 | 0 | 0 | 0 | 2 |
| | Speech rate | 2 | 1 | 0 | 0 | 2 | 0 | 8 |
| | Speech patterns | 0 | 0 | 0 | 0 | 2 | 0 | 4 |
| | Weighed estimate | 0.55 | 0.50 | 0.18 | 0.43 | 1.68 | 0.80 | 40 |

TABLE No2

| External | | |
|---|---|---|
| parameters | Objects of comparison | Values/data |
| Statistical | Full face, | Key points, their coordinates, angles |
| external | Half-faced, | between them, |
| | 3D model of head, | Vectors of objects' contour, Coordinates of |
| | 3D model of face, | mesh points (based on different |
| | Facial asymmetry, | mathematical methods and conversions), |
| | Physiognomic characteristics | Parameters by method of own faces |
| | (shape of face, nose, eyes, ears, | (eigenfaces), |
| | lips, chin, cheekbones, forehead, | Ratio of values, distances and volumes, |
| | cheeks, eyebrows, superciliary | Combinations of characteristics (patterns) |
| | archs, wrinkles and folds, teeth, | |
| | gums, zones of pilosis, shape and | |
| | position of mimic folds/wrinkles, | |
| | facial asymmetry) | |
| | Body parts (shape of head, necks, | |
| | shoulders, corpus, extremities, | |
| | fingers and palms, nails, elbows | |
| | and knees) | |
| | Body and its parts | |
| Dynamic | Position and movements of body | Directions of dynamic, |
| external | and its parts, | Change frequency of direction, |
| | Eye movements, | Speed and acceleration of movements, |
| | Physiognomy and mime, mimic | Movement stops (pauses) with regard to |
| | deformations of facial parts, | direction, |
| | Mimic wrinkles and folds, | Combinations of characteristics (patterns) |
| | Mime's activity of different facial | |
| | parts, | |
| | Mimic asymmetry. | |
| Speech | Voice, | Main frequencies, formants, densities of |
| | Speech and sounds accompanying | frequencies and their distributions, |
| | speech | characteristics of transitions between |
| | Pauses, | sentences and while changing the breath, |
| | Intonation and loudness | timbre and tonal sound, instantaneous |
| | | values and levels of speech signal; the |
| | | duration of continuous existence of different |
| | | levels; duration and frequency of pauses; |
| | | the distribution of maximum levels by |
| | | frequency; the distribution of current and |
| | | average power; the distribution of power |
| | | spectral density; |
| | | dynamic range and peak-factor, |
| | | the distribution of the main phonation |
| | | frequency, spectral distribution of formant, |
| | | combinations of characteristics (speech |
| | | patterns). |

1. (canceled)

2. (canceled)

3. (canceled)

4. (canceled)

5. (canceled)

6. A method for determining a psyche type of an tested individual, the method comprising:
forming a sampling of individuals with different known types of psyche;
selecting external parameters;
measuring the external parameters of the tested individual and the external parameters of individuals from sampling;
comparing the external parameters of the tested individual with the external parameters of individuals from sampling; and
relating the psyche type of the tested individual to a psyche type of an individual from the sampling based on a similarity by the external parameters of the tested individual and the individual from the sampling.

7. The method of claim 6, wherein the similarity by the external parameters is equal or greater than a predetermined value.

8. The method of claim 6, wherein the psyche type of the tested individual is determined if the similarity by the external parameters is equal or greater than a predetermined level.

9. The method of claim 6, wherein the different known types of psyche are defined on the basis of characteristics: ethics-logic, intuition-sensation, extraversion-introversion, and rationality-irrationality.

10. The method of claim 6, wherein the different known types of psyche are defined on the basis of characteristics: ethics-logic, intuition-sensation, extraversion-introversion, and rationality-irrationality and a type of temperament.

11. The method of claim 6, wherein the external parameters include one or more of the following: voice, speech rate, speech pattern, shape of a human body part, position of the human body part, and movement of the human body part.

12. The method of claim 6, wherein the similarity is determined based on similarities of each of the external parameters and weights assigned to the each of the external parameters.
